(19) United States
(12) Patent Application Publication
(10) Pub. No.: US 2018/0079778 A1
(43) Pub. Date: Mar. 22, 2018

(54) METHOD OF REMOVING PROTEIN AGGREGATE

(71) Applicant: SHOWA DENKO K.K., Tokyo (JP)

(72) Inventors: Yuzuru KOKIDO, Tokyo (JP); Natsuno MATSUI, Tokyo (JP); Hirobumi AOKI, Tokyo (JP); Tadashi YONEDA, Tokyo (JP)

(73) Assignee: SHOWA DENKO K.K., Tokyo (JP)

(21) Appl. No.: 15/569,424

(22) PCT Filed: Apr. 28, 2016
(86) PCT No.: PCT/JP2016/063937

§ 371 Date: Oct. 26, 2017

(30) Foreign Application Priority Data
Apr. 30, 2015 (JP) 2015-093649

(51) Int. Cl. C07K 1/18; A61K 39/395; C07K 16/06

(52) U.S. Cl. CPC C07K 1/18; C07K 16/065; A61K 39/39591

(57) ABSTRACT

Provided is a method of removing a protein aggregate which can obtain a useful protein monomer in a high yield and a high purity as a raw material of a pharmaceutical product or the like. The method of removing a protein aggregate includes: a step of making the monomer of a protein and the aggregate of proteins adsorb to the column by making a solution containing a monomer of a protein and an aggregate of proteins pass through a column containing a hard porous polymer self-supporting structure to which a strong cation exchange group is fixed; and a step of making a mobile phase consisting of a mixed solution of a buffer solution and an ionic buffer solution pass through the column to which the monomer of a protein and the aggregate of proteins are adsorbed, to selectively elute the monomer of a protein.

### TECHNICAL FIELD

The present invention relates to a method of removing a protein aggregate from a solution which contains a protein monomer and a protein aggregate.

Priority is claimed on Japanese Patent Application No. 2015-093649, filed Apr. 30, 2015, the content of which is incorporated herein by reference.

### BACKGROUND ART

In recent years, treatment in which proteins such as immunoglobulin or cytokine as pharmaceuticals has been widely used. These proteins are called biopharmaceuticals of which the market has also been widely expanded. Among these, antibody pharmaceuticals using immunoglobulin have already been used as therapeutic medicines for autoimmune diseases or cancer. It is expected that the market of antibody pharmaceuticals might be further expanded in the future.

It is necessary to highly purify proteins for pharmaceuticals from a cell culture liquid containing a large amount of impurities. As the purification method, chromatography or the like can be used.

However, in some cases, purified proteins form an aggregate such as a dimer or a trimer and become impurities. There is a concern that the formation of protein aggregates may cause harmful effects, such as decrease in medicinal effects or expression of immunogenicity, as pharmaceutical products. For this reason, it is desirable to remove protein aggregates which are impurities to the extent to which there is no harmful effect.

Based on the above, many methods for removing a protein aggregate through chromatography for the purpose of purifying proteins for pharmaceuticals, in particular, antibody proteins or the like have been disclosed.

Examples of the methods for removing a protein aggregate through chromatography include a method of singly using anion exchange chromatography, cation exchange chromatography, hydrophobic chromatography, or the like, a method of using a plurality of these kinds of chromatography, and mixed-mode chromatography in which a plurality of these kinds of chromatography are mixed. All of these methods are methods performed through chromatography using a bead-like porous polymer carrier. In order to separate both a protein aggregate and a protein monomer from each other using the slight difference of interaction therebetween, precise control of the separation conditions and exhibition of excellent separation capacity are required. However, it is known that, in chromatography in which a bead-like porous polymer carrier is used, the separation capacity is deteriorated at a high flow rate. Accordingly, in the chromatography in which a bead-like porous polymer carrier is used, it is difficult to promptly separate a protein aggregate and a protein monomer from each other while making the protein aggregate and the protein monomer pass through a solution at a high flow rate.

As the method of removing an aggregate at a high flow rate, a method for purifying an antibody monomer using a porous membrane to which an anion exchange group is fixed is disclosed (for example, refer to Patent Document 1). In this method, a hollow porous membrane module with a 2 mL capacity is passed through a solution at a flow rate of 2 mL/min. This flow rate is not different from a flow rate of 1 mL/min per volume which is used in a commercially available bead-filling type anion exchange chromatography column (1 mL of column solution). That is, in this method, it is impossible to realize prompt separation of a protein aggregate and a protein monomer by making the protein aggregate and the protein monomer pass through a solution, and therefore, a new method has been required.

In addition, chromatography in which a porous self-supporting structure is used has been known (for example, refer to Patent Document 2). This porous self-supporting structure is produced from a mixture of a vinyl monomer and polyvinyl, has a number of fine pores which are uniform in a specific range, and contains a polymer into which a functional group such as an ion exchange group, a hydrophobic group, or an affinity ligand is introduced. Such a porous self-supporting structure is excellent in physical strength compared to a bead-like porous polymer carrier or a porous membrane, and of which the back pressure is low at a high flow rate. Accordingly, it is considered that the porous self-supporting structure can separate a target substance from another by making the substances pass through a solution at a high flow rate. However, in the related art, a method of removing a protein aggregate through chromatography in which a porous self-supporting structure is used has not been known.

Patent Document 1: Japanese Unexamined Patent Application, First Publication No. 2010-241761

Patent Document 2: Published Japanese Translation No. 2002-505428 of the PCT International Publication

### SUMMARY OF THE INVENTION

The present invention has been made in consideration of the above-described circumstances, and an object of the present invention is to provide a method of removing a protein aggregate which can obtain a useful protein monomer in a high yield and a high purity as a raw material of a pharmaceutical product. More specifically, an object of the present invention is to provide a method of removing a protein aggregate which can remove the protein aggregate contained in a protein at a high speed and to obtain a high yield of a protein with a high purity of a protein monomer.

The present inventors conducted extensive studies on the method of removing a protein aggregate in order to solve the above-described problems. As a result, they have found that, even in a case where the flow rate of a mobile phase is fast with respect to the volume of a column containing a hard porous polymer self-supporting structure to which a strong cation exchange group is fixed, it is possible to remove the protein aggregate at a high speed and to obtain a high yield of a protein with a high purity of a protein monomer, by performing chromatography using the column, and have completed the present invention.

[1] A method of removing a protein aggregate, including:

a step of making a monomer of a protein and an aggregate of proteins adsorb to a column containing a hard porous polymer self-supporting structure to which a strong cation exchange group is fixed by making a solution containing the monomer of the protein and the aggregate of proteins pass through the column; and a step of making a mobile phase consisting of a mixed solution of a buffer solution and an ionic buffer solution pass through the column to which the monomer of a protein and the aggregate of proteins are adsorbed, to selectively elute the monomer of a protein.

[2] The method of removing a protein aggregate according to [1],

in which the concentration of the buffer solution is 1 mM to 100 mM.

[3] The method of removing a protein aggregate according to [1] or [2],

in which the pH of the buffer solution is 2 to 9.

[4] The method of removing a protein aggregate according to any one of [1] to [3],

in which the concentration of inorganic salts in the mixed solution of the buffer solution and the ionic buffer solution is 50 mM to 250 mM.

[5] The method of removing a protein aggregate according to any one of [1] to [4],

in which monomers constituting the hard porous polymer self-supporting structure are methacrylates or acrylates.

[6] The method of removing a protein aggregate according to any one of [1] to [5],

in which the thickness of the hard porous polymer self-supporting structure in a direction in which a solution passes through is 1 mm to 100 mm.

[7] The method of removing a protein aggregate according to any one of [1] to [6],

in which the flow rate of the mobile phase is greater than or equal to 2 CV/min.

### Advantageous Effects of Invention

According to the method of removing a protein aggregate of the present invention, it is possible to obtain an industrially and economically useful protein in a high yield and a high purity as a raw material of a pharmaceutical product or the like. More specifically, it is possible to provide a method of removing a protein aggregate contained in a protein at a high speed and for obtaining a high yield of a protein with a high purity of a protein monomer.

### BRIEF DESCRIPTION OF THE DRAWINGS

FIG. 1 shows a flow-through portion (T1), elution portion (T2) using a mixed solution of buffer solutions which are used for equilibration, and an elution portion (T3) using an ionic buffer solution, in a chromatogram.

FIG. 2 is a flow path diagram of a device which is used for calculating a recovery rate in Examples.

FIG. 3 is a flow path diagram of a device which is used for calculating purity in Examples.

### DETAIL DESCRIPTION OF THE INVENTION

Hereinafter, an embodiment of a method of removing a protein aggregate of the present invention will be described.

The present embodiment is specifically described for better understanding of the gist of the invention and does not limit the present invention unless otherwise specified.

### [Method of Removing Protein Aggregate]

The method of removing a protein aggregate of the present invention is a method including: a step of making a monomer of a protein (hereinafter, referred to as a “protein monomer”) and an aggregate of proteins (hereinafter, referred to as a “protein aggregate”) adsorb to a column containing a hard porous polymer self-supporting structure to which a strong cation exchange group is fixed by making a solution containing the protein monomer and the protein aggregate pass through the column; and a step of making a mobile phase consisting of a mixed solution of a buffer solution and an ionic buffer solution pass through the column to which the protein monomer and the protein aggregate are adsorbed, to selectively elute the protein monomer. Accordingly, the protein aggregate is removed.

The protein monomer represents one molecule of a protein. The protein aggregate is a protein complex in which protein monomers are reversibly or irreversibly adsorbed and aggregated through a hydrophobic interaction, an electrostatic interaction, and other interactions.

### <Strong Cation Exchange Group>

A strong cation exchange group is a cation exchange group with a high ion strength. The strong cation exchange group is preferably an exchange group showing strong acidity. Specific examples of the strong cation exchange group include a sulfo group. The sulfo group may be held by a hard porous polymer self-supporting structure through a linker such as an alkyl group or a hydroxy alkyl group.

### <Hard Porous Polymer Self-Supporting Structure>

The hard porous polymer self-supporting structure is a structure of a polymer obtained through polymerization of monomers having at least two polymerizable portions or at least two monomers. In addition, the hard porous polymer self-supporting structure is porous and is a porous body of which holes have uniform pore diameter distribution over the entirety.

The hard porous polymer self-supporting structure forms an integral structure with a shape such as a plate shape, tubular shape, and a cylindrical shape.

The thickness of the hard porous polymer self-supporting structure in a direction in which a solution passes through is preferably 1 mm to 100 mm, more preferably 2 mm to 70 mm, still more preferably 3 mm to 60 mm, and most preferably 3 mm to 50 mm.

By making the thickness of the hard porous polymer self-supporting structure be greater than or equal to 1 mm, the hard porous polymer self-supporting structure has a sufficient mechanical strength. By making the thickness of the hard porous polymer self-supporting structure be less than or equal to 100 mm, it is possible to prevent increase of pump pressure while maintaining the pressure so as not to destroy the hard porous polymer self-supporting structure.

Specific examples of the hard porous polymer self-supporting structure include CIMac (registered trademark) SO3-0.1 Analytical column, CIM (registered trademark) SO3 DISK, CIM SO3-1 Tube Monolithic column, CIM SO3-8f Tube Monolithic column, CIM SO3-80 Tube Monolithic column, CIM SO3-800 Tube Monolithic column, CIM SO3-8000 Tube Monolithic column, CIMMultus (registered trademark) SO3-1 Advanced Composit column, CIMMultus SO3-8 Advanced Composit column, CIMMultus SO3-80 Advanced Composit column, CIMMultus SO3-800 Advanced Composit column, and CIMMultus SO3-8000 Advanced Composit column.

All of these hard porous polymer self-supporting structures are copolymers of glycidyl methacrylate and ethylene dimethacrylate.

These hard porous polymer self-supporting structures have mechanical strength which can self-support the shapes thereof when being left to stand. That is, the physical structure of the hard porous polymer self-supporting structure is different from that of a bead-like porous polymer carrier or a porous membrane.

As a monomer for constituting the hard porous polymer self-supporting structure, a monomer having a strong cation exchange group or a monomer having a functional group which becomes a group of the strong cation exchange group, and examples thereof include a polyvinyl monomer or a monovinyl monomer.

As the polyvinyl monomer, divinylbenzene, divinyl naphthalene, divinyl pyridine, methacrylates, acrylates, vinyl esters, vinyl ethers such as divinyl ether, alkylene bisacrylamides such as ethylene bisacrylamide or propylene bisacrylamide, or methacrylamides, and mixtures thereof can be used.

As the methacrylates, glycidyl methacrylate, alkylene dimethacrylates such as ethylene glycol dimethacrylate or propylene glycol dimethacrylate, hydroxy alkyl methacrylates such as hydroxy ethyl methacrylate or hydroxy propyl methacrylate, pentaerythritol di-, tri-, or tetramethacrylate, trimethylol propane trimethacrylate, or acrylate are used.

As the acrylates, ethylene glycol diacrylates, or pentaerythritol di-, tri-, or tetraacrylate are used.

Examples of the monovinyl monomer include styrene, substituted styrene (however, a substituted group includes chloromethyl group, an alkyl group having 1 to 18 carbon atoms, hydroxyl group, a t-butyloxycarbonyl group, a halogen group, a nitro group, an amino group, a protected hydroxyl group, or a protected amino group), vinyl naphthalene, acrylic acid esters, methacrylic acid esters, vinyl acetate, pyrrolidone, and mixtures thereof. An example of the methacrylic acid ester includes glycidyl methacrylate.

As the monomer for constituting the hard porous polymer self-supporting structure, methacrylates or acrylates are preferable, glycidyl methacrylate or alkylene dimethacrylates are more preferable, and one which is obtained by modifying a copolymer of glycidyl methacrylate and ethylene glycol dimethacrylate with a compound having a sulfo group is still more preferable.

The copolymer of glycidyl methacrylate and ethylene glycol dimethacrylate is produced from a mixture of glycidyl methacrylate and ethylene glycol dimethacrylate in the presence of porogen and a polymerization initiator.

Porogen is an additive substance for forming many holes, and as porogen, different kinds of materials such as aliphatic hydrocarbons, aromatic hydrocarbons, esters, alcohols, ketones, ethers, soluble polymer solution, and mixtures thereof can be used. Porogen is preferably normal hexane.

As the polymerization initiator, a free radical-generating initiator is used. Specifically, azo compounds such as azobisisobutyronitrile or 2,2′-azobis(isobutylamide)dihydrate, and peroxide such as benzoyl peroxide or peroxide dipropyl dicarboxylic acid ester can be used. If different kinds of polymerization initiators are used, it is possible to form different forms of hole structures.

The amount of the polymerization initiator is preferably 0.5 mass % to 4 mass % with respect to the weight of monomers.

When producing the copolymer of glycidyl methacrylate and ethylene glycol dimethacrylate, a soluble polymer may be added as porogen. If the soluble polymer is added, a larger number of hole structures are formed. The amount of soluble polymer is preferably 10 mass % to 40 mass % with respect to the total mass of the copolymer.

It is preferable that the mixture of glycidyl methacrylate and ethylene glycol dimethacrylate which contains porogen or a polymerization initiator is degassed using inert gas such as nitrogen or argon before being placed in a mold. The mold is preferably sealed in order to prevent air contamination.

The above-described polymerization of monomers can be performed by, for example, applying heat at a temperature of 50° C. to 90° C. for 40 hours to 50 hours.

After the polymerization, a tube is cleaned to remove a solvent or a soluble polymer which has been used as porogen. As the solvent for cleaning, methanol, ethanol, benzene, toluene, acetone, tetrahydrofuran, or the like can be used. The cleaning step may be repeated a plurality of times.

When modifying a copolymer, it is possible to introduce a strong cation exchange group using a disulfate 1,4-dioxane mixture.

### <Column>

A column includes a hard porous polymer self-supporting structure to which a strong cation exchange group is fixed. That is, the column may be constituted of only a hard porous polymer self-supporting structure forming a shape such as a plate shape, tubular shape, and a cylindrical shape, or may be obtained by housing (holding) a predetermined amount of hard porous polymer self-supporting structure in a cylindrical container.

The size (volume) of a column is not particularly limited and can be appropriately adjusted according to the amount of protein monomer and protein aggregate to be adsorbed.

### <Protein>

Protein from which a protein aggregate can be removed through a method of removing a protein aggregate of the present invention is not particularly limited. Examples of protein from which a protein aggregate can be removed through the method of removing a protein aggregate of the present invention include immunoglobulin such as IgG, IgA, and IgM, Cytokines such as interleukin, chemokine, interferon, G-CSF, erythropoietin, EGF, FGF, PDGF, HGF, TNF-α, THF-β, adipokine, and NGF, protein hormones such as human growth hormone, insulin, and glucagon, a blood coagulation factor, albumin, lysozyme, RNaseA, and cytochrome c.

The protein aggregate is a protein complex in which protein monomers are reversibly or irreversibly adsorbed and aggregated through a hydrophobic interaction, an electrostatic interaction, and other interactions.

### <Adsorption>

A solution (hereinafter, this solution is called a “protein solution”) is prepared by dissolving a protein monomer and a protein aggregate in a buffer solution (a first buffer solution), and this protein solution is passed through a column. Accordingly, it is possible to make proteins (protein monomer and protein aggregate) contained in this protein solution be adsorbed to the column including a hard porous polymer self-supporting structure.

### <Buffer Solution >

As the buffer solution, for example, a phosphate buffer solution, a citrate buffer solution, a tris(trishydroxymethylaminomethane) buffer solution, an acetate buffer solution, and a borate buffer solution can be used. Among these, a phosphate buffer solution, a citrate buffer solution, and a tris buffer solution are preferable in view of a pH range which has a buffer capacity and is used.

The concentration of the buffer solution is not particularly limited, but is preferably 1 mM to 100 mM, is more preferably 2 mM to 50 mM, and is still more preferably 5 mM to 30 mM.

The pH of the buffer solution is not particularly limited as long as the pH of the buffer solution is within a range in which protein can be adsorbed and eluted. However, the pH of the buffer solution is preferably pH 2 to pH 9, is more preferably pH 3 to pH 8, and is still more preferably pH 4 to pH 7.5.

The concentration of proteins in a protein solution is preferably 0.01 mg/mL to 10 mg/mL, is more preferably 0.1 mg/mL to 5 mg/mL, and is still more preferably 0.2 mg/mL to 3 mg/mL.

By making the concentration of proteins be within the above-described range, it is possible to make all the proteins contained in the protein solution be adsorbed to a column containing a hard porous polymer self-supporting structure.

It is preferable to perform equilibration by making a buffer solution pass through the column before making proteins be adsorbed to the column.

As the buffer solution used for the equilibration of a column, a buffer solution of which the type, the concentration, and the pH are the same as those of a buffer solution (a first buffer solution) dissolving proteins can be used.

The amount of buffer solution required for the equilibration of a column is not particularly limited, but is preferably greater than or equal to 1 CV (multiple of column volume), is more preferably greater than or equal to 2 CV, and is still more preferably greater than or equal to 4 CV.

The flow rate of a protein solution in a column is not particularly limited as long as proteins can be adsorbed to the column, but is preferably 2 CV/min to 12.5 CV/min, and is more preferably 2.5 CV/min to 5 CV/min.

When making proteins be adsorbed to the column, the temperature of the column and the protein solution is not particularly limited, but is preferably 2° C. to 50° C., is more preferably 4° C. to 40° C., and is still more preferably 8° C. to 30° C. By making the temperature of the column and the protein solution be within the range, it is possible to prevent freezing of the protein solution and destroy of proteins.

### <Elution of Protein Monomer>

A mixed solution is prepared by mixing an ionic buffer solution and the above-described first buffer solution at an appropriate ratio.

It is possible to selectively elute a protein monomer by making a mobile phase pass through the column to which proteins are adsorbed, using this mixed solution as the mobile phase.

### <Ionic Buffer Solution>

The ionic buffer solution is a solution prepared by dissolving an ionic solute into a second buffer solution. The type, the concentration, and the pH of the second buffer solution in which inorganic salts are dissolved may be the same as or different from those of the first buffer solution in which proteins are dissolved. It is preferable that the second buffer solution used to prepare the ionic buffer solution is the same as the above-described first buffer solution.

The solute used in an ionic buffer solution is not particularly limited as long as it is possible to selectively elute a protein monomer, but is preferably ammonium sulfate, sodium chloride, or sodium citrate.

The concentration of the solute in the above-described mixed solution of the first buffer solution and the ionic buffer solution is preferably 50 mM to 250 mM, is more preferably 75 mM to 225 mM, and is still more preferably 100 mM to 200 mM.

The flow rate of a mobile phase is not particularly limited as long as it is possible to selectively elute a protein monomer which is adsorbed to a column, but is preferably greater than or equal to 2 CV/min, is more preferably 2 CV/min to 12.5 CV/min, and is still more preferably 2.5 CV/min to 5 CV/min.

When making a mobile phase pass through a column, the concentration the column and the mobile phase is not particularly limited, but is preferably 2° C. to 50° C., is more preferably 4° C. to 40° C., and is still more preferably 8° C. to 30° C. By making the concentration of the column and the mobile phase be within the range, it is possible to prevent freezing of the protein solution and destroy of proteins.

By making a mobile phase pass through a column using only the above-described ionic buffer solution as the mobile phase after eluting a protein monomer, it is also possible to elute a protein aggregate which has been adsorbed to the column.

By making the above-described buffer solution pass through the column again after eluting the protein aggregate, it is possible to regenerate the column.

According to the method of removing a protein aggregate of the present invention, it is possible to remove the protein aggregate contained in a protein at a high speed and to obtain a high yield of a protein with a high purity of a protein monomer, by selectively eluting only the protein monomer from a column after making the protein monomer and the protein aggregate be adsorbed to the column containing a hard porous polymer self-supporting structure to which a strong cation exchange group is fixed. The obtained protein monomer is useful as a raw material of pharmaceutical products or the like.

In addition, the method of removing a protein aggregate of the present invention is an industrially and economically excellent method since it is possible to remove the protein aggregate contained in a protein at a high speed and it is possible to easily regenerate the column.

### EXAMPLES

Hereinafter, the present invention will be described more specifically using Examples and Comparative Examples. However, the present invention is not limited to the following Examples.

In Examples and Comparative Examples, the recovery rate, the purity, the aggregate content are defined as follows.

### [Recovery Rate]

A recovery rate is defined as the ratio of immunoglobulin G (hereinafter, abbreviated as “IgG”) monomers, which are eluted and purified, to the total amount of IgG which is adsorbed to a column, that is, the sum of IgG monomers and IgG aggregates.

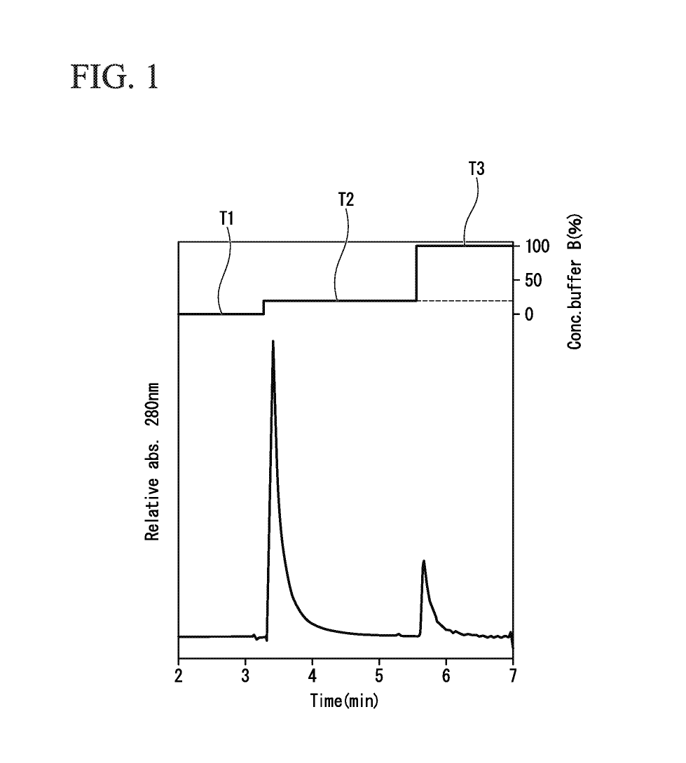

Specifically, on the chromatogram, a value which is obtained by dividing the area of an elution portion using a mixed solution of an ionic buffer solution and a buffer solution which is used for equilibration of a column, by the sum of the area of a flow-through portion, the area of an elution portion using a mixed solution, and the area of an elution portion using an ionic buffer solution is regarded as recovery rate. The flow-through portion (T1), the elution portion (T2) using a mixed solution buffer solutions used for equilibration of a column, and the elution portion (T3) using an ionic buffer solution are as shown in FIG. 1.

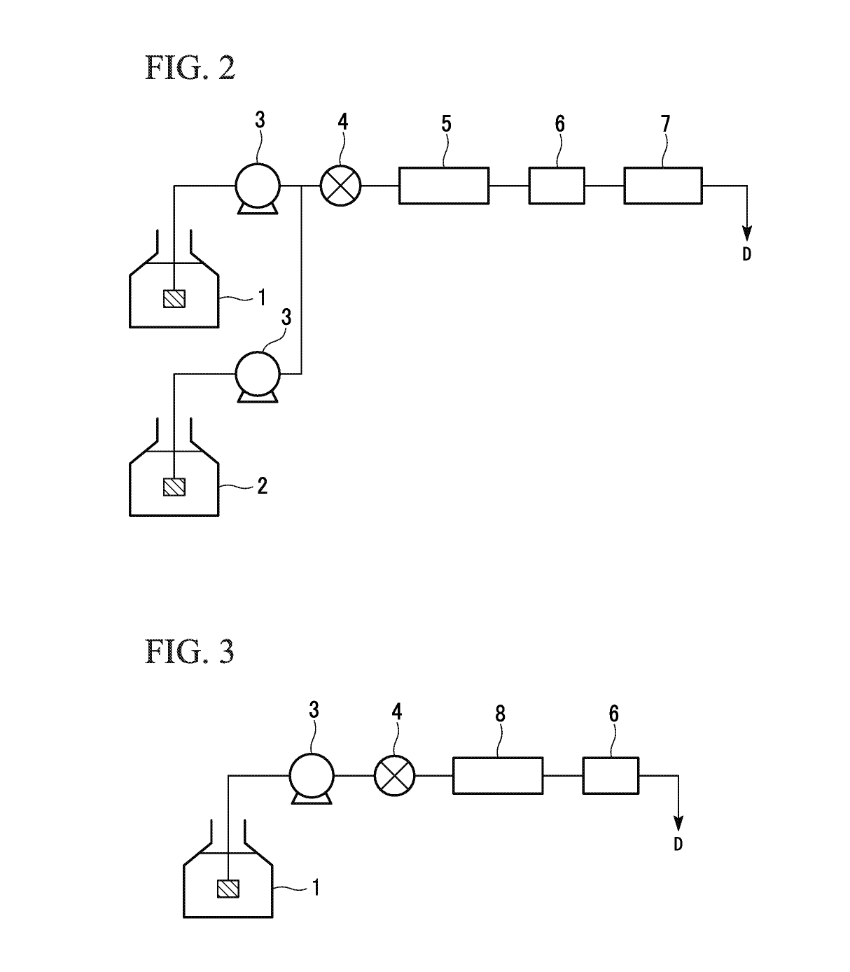

When creating a chromatogram, a liquid chromatograph system controller SCL-10 AVP manufactured by Shimadzu Corporation is used. In this device, a buffer solution and an ionic buffer solution are connected to an autosampler through a liquid feeding pump, and therefore, it is possible to control the concentration of the solutions flowing in a column. A flow path diagram is shown in FIG. 2. In FIG. 2, the reference numeral 1 represents a buffer solution, the reference numeral 2 represents an ionic buffer solution, the reference numeral 3 represents a liquid feeding pump, the reference numeral 4 represents an autosampler, the reference numeral 5 represents a fractionating column, the reference numeral 6 represents a PDA detector, and the reference numeral 7 represents a fraction collector. The unit is %.

### [Purity]

Purity is defined as the ratio of an IgG monomer with respect to the total amount of IgG in a sample which has been collected, that is, purity is defined as the sum of an IgG monomers and IgG aggregates.

Specifically, the purity is obtained by measuring the proportion of the IgG monomers through size exclusion chromatography using a column (shodex (registered trademark) KW403-4F) manufactured by SHOWA DENKO K.K. as an analysis column. The value obtained by dividing the peak area of monomers on the chromatogram by the area of the total amount of IgG is regarded as purity. The area of the total amount of IgG is regarded as the sum of the peak area of aggregates and the peak area of monomers. When creating the chromatogram, a liquid chromatograph feed unit LC20-AT is used. A flow path diagram is shown in FIG. 3. In FIG. 3, the agreement 1 represents a buffer solution, the reference numeral 3 represents a liquid feeding pump, the reference numeral 4 represents an autosampler, the reference numeral 6 represents a PDA detector, and the reference numeral 8 represents an analysis column. The unit is %.

### [Aggregate Content]

Aggregate content is defined as 100—purity (%), when a state in which no aggregate is contained at all is set to 100. The purity (%) is calculated as an area ratio in a chromatogram, as above-mentioned.

### Example 1

### (1) Equilibration of Column

CIM (registered trademark) SO3 DISK (thickness: 3 mm, diameter: 12.0 mm, porosity: greater than or equal to about 60 v/v %) manufactured by BIA separations was used as a column. This column was equilibrated by making greater than or equal to 5 CV of a 20 mM citrate buffer solution at a pH of 5.3 pass through this column. CIM SO3 DISK is a modified product of a copolymer of glycidyl methacrylate and ethylene glycol dimethacrylate, and is a hard porous polymer self-supporting structure in which a sulfo group is held by a part of glycidyl methacrylate.

### (2) Adsorption of IgG

A protein solution of which the concentration of IgG is 3 mg/mL was prepared by dissolving IgG (manufactured by Sigma-Aldrich) in a 20 mM citrate buffer solution with a pH of 5.3. The aggregate content of this solution was about 15—purity %.

50 μL of this protein solution was injected into the column which had been equilibrated in (1), and proteins contained in the protein solution were adsorbed to the column. At this time, the amount of load of proteins per 1 mL of column volume was 450 μg. The flow rate of the protein solution in the column was set to 12 CV/min.

### (3) Elution of IgG Monomer

A mixed solution was prepared by mixing a 20 mM citrate buffer solution at a pH of 5.3 and a 1M of NaCl solution such that the concentration of NaCl became 170 mM. The 1M NaCl solution was a solution obtained by dissolving NaCl in the 20 mM citrate buffer solution at a pH of 5.3.

IgG monomers were eluted by making 6 ml of this mixed solution pass through the column to which proteins were adsorbed. The flow rate of the solution was set to 12 CV/min.

### (4) Elution of IgG Aggregate

Thereafter, IgG aggregates were eluted by making 6 ml of the 1M NaCl solution pass through the column. The flow rate of the solution was set to 12 CV/min.

In Example 1, the temperature of the column, the protein solution, and the mobile phase was set to 25° C., and the pressure of the column was set to be within a range of not exceeding the maximum allowable pressure of 1.8 MPa.

### Example 2

IgG monomers were selectively eluted similarly to Example 1 except that the concentration of NaCl was set to 200 mM in (3) of Example 1.

### Example 3

IgG monomers were selectively eluted similarly to Example 1 except that the concentration of NaCl was set to 140 mM in (3) of Example 1.

### Example 4

### (1) Equilibration of Column

CIMMultus (registered trademark) SO3-1 Tube (thickness: 6 mm, outer diameter: 18.6 mm, inner diameter: 6.7 mm, Length: 4.2 mm, porosity: greater than or equal to about 60 v/v %) manufactured by BIA separations was used as a column. This column was equilibrated similarly to Example 1.

CIMMultus (registered trademark) SO3-1 Tube is a modified product of a copolymer of glycidyl methacrylate and ethylene glycol dimethacrylate, and is a hard porous polymer self-supporting structure in which a sulfo group is held by a part of glycidyl methacrylate. In addition, holes in the center of the column is blocked by the structure of the device, and therefore, the injected solution does not pass through the holes in the center thereof as it is.

### (2) Adsorption of IgG

A protein solution of which the concentration of IgG is 3 mg/mL was prepared similarly to Example 1. The aggregate content of this solution was about 20—purity %.

100 μL of this protein solution was injected to the column which had been equilibrated in (1), and proteins contained in the protein solution were adsorbed to the column. At this time, the amount of load of proteins per 1 mL of column volume was 300 μg. The flow rate of the protein solution in the column was set to 5 CV/min.

### (3) Elution of IgG Monomer

A mixed solution was prepared by mixing a 20 mM citrate buffer solution at a pH of 5.3 and a 1M of NaCl solution such that the concentration of NaCl became 150 mM. The 1M NaCl solution was a solution obtained by dissolving NaCl in the 20 mM citrate buffer solution at a pH of 5.3.

IgG monomers were eluted by making 7.5 ml of this mixed solution pass through the column to which proteins were adsorbed. The flow rate of the solution was set to 5 CV/min.

### (4) Elution of IgG Aggregate

Thereafter, IgG aggregates were eluted by making 7.5 ml of the 1M NaCl solution pass through the column. The flow rate of the solution was set to 5 CV/min.

### Example 5

IgG monomers were selectively eluted similarly to Example 4 except that the concentration of NaCl was set to 100 mM in (3) of Example 4.

### Example 6

IgG monomers were selectively eluted similarly to Example 4 except that the concentration of NaCl was set to 130 mM in (3) of Example 4.

### Example 7

IgG monomers were selectively eluted similarly to Example 4 except that the amount of load of proteins per 1 mL of column volume was 10 mg in (2) to (4) of Example 4.

### Example 8

IgG monomers were selectively eluted similarly to Example 4 except that each flow rate of the flow rate of the protein solution in the column in (2) of Example 4, the flow rate of the solution in (3) of Example 4, and the flow rate of the solution in (4) of Example 4 was 2 CV/min.

### Example 9

IgG monomers were selectively eluted similarly to Example 4 except that each flow rate of the flow rate of the protein solution in the column in (2) of Example 4, the flow rate of the solution in (3) of Example 4, and the flow rate of the solution in (4) of Example 4 was 10 CV/min.

### Comparative Example 1

A protein solution which was the same as that used in Example 1 was made to pass through a column containing a hard porous polymer self-supporting structure to which an anion exchange group is fixed, that is, CIMMultus (registered trademark) QA-1 Advanced Composit column (width: 6 mm, outer diameter: 18.6 mm, inner diameter: 6.7 mm, length: 4.2 mm, porosity: greater than or equal to about 60 v/v %) manufactured by BIA separations.

CIMMultus (registered trademark) QA-1 Advanced Composit column is a modified product of a copolymer of glycidyl methacrylate and ethylene glycol dimethacrylate, and is a hard porous polymer self-supporting structure in which a quaternary ammonium group is held by a part of glycidyl methacrylate.

However, IgG was collected as flow-through without being adsorbed to the column. For this reason, it was impossible to improve the purity of IgG monomers.

### Comparative Example 2

IgG monomers were selectively eluted similarly to Example 6 except that HiTrap SP FF (average particle diameter of 90 μm) manufactured by GE Healthcare was used as porous polymer beads constituting the column and the flow rate of the protein solution in the column was set to 1 CV/min.

### Comparative Example 3

A protein solution was injected to a column similarly to Example 6 except that HiTrap SP FF (average particle diameter of 90 μm) manufactured by GE Healthcare was used as porous polymer beads constituting the column and the flow rate of the protein solution in the column was set to 2 CV/min. As a result, it was impossible to operate the beads due to increased pressure which exceeded an allowable pressure.

Various conditions and the evaluation results of Examples 1 to 9 and Comparative Examples 1 to 3 are shown in Table 1.

TABLE 1

| | | | | | | Flow | | | |
|---|---|---|---|---|---|---|---|---|---|
| | Column | | | | Amount of load | rate during | | | |
| | | Strong | | | of IgG per 1 mL | elution of | Concentration of | Recovery | |
| | | cationic | Constituent | Thickness | of column | monomer | NaCl during Elution | rate | Purity |
| | Type | group | monomer | [mm] | volume [mg] | [CV/min] | of monomer [mM] | [%] | [%] |
| Example 1 | CIM ® | Sulfo group | Glycidyl | 3 | 0.45 | 12 | 170 | 56 | 98 |
| Example 2 | SO3 | Sulfo group | methacrylate | 3 | 0.45 | 12 | 200 | 80 | 99 |
| Example 3 | DISK | Sulfo group | Ethylene glycol | 3 | 0.45 | 12 | 140 | 29 | Greater |
| | | | dimethacrylate | | | | | | than or |
| | | | | | | | | | equal to |
| | | | | | | | | | 99 |
| Example 4 | CIM | Sulfo group | | 6 | 0.3 | 5 | 150 | 69 | 96 |
| Example 5 | Multus ® | Sulfo group | | 6 | 0.3 | 5 | 100 | 21 | 99 |
| Example 6 | SO3-1 | Sulfo group | | 6 | 0.3 | 5 | 130 | 47 | 99 |
| Example 7 | Tube | Sulfo group | | 6 | 10 | 5 | 130 | 67 | 94 |
| Example 8 | | Sulfo group | | 6 | 0.3 | 2 | 150 | 50 | 99 |
| Example 9 | | Sulfo group | | 6 | 0.3 | 10 | 130 | 62 | 98 |
| Comparative | QA-1 | None (functional | | 6 | 0.3 | 5 | 0 | 100 | 85 |
| Example 1 | Tube | group: quaternary | | | | | | | |
| | | ammonium | | | | | | | |
| | | group) | | | | | | | |
| Comparative | HiTrap | Sulfo group | Beads | 0.09 | 0.3 | 1 | 130 | 63 | 94 |
| Example 2 | SP FF | | | | | | | | |
| Comparative | HiTrap | Sulfo group | Beads | 0.09 | 0.3 | 2 | — | — | — |
| Example 3 | SP FF | | | | | | | | |

From the results in Table 1, the purity of IgG monomers was greater than or equal to 94% in Examples 1 to 9.

In contrast, in Comparative Example 1, the purity of IgG monomers was 85% while the recovery rate of IgG monomers was 100%.

In Comparative Example 2, the recovery rate and the purity which are the same as those in Examples were achieved by setting the flow rate of the protein solution to a low value. However, in Comparative Example 3 in which porous polymer beads are used, it was impossible to increase the flow rate to greater than or equal to 2 CV/min from the viewpoint of allowable pressure. Therefore, the aspects of Examples are excellent in that it was possible to perform separation at a higher speed.

### REFERENCE SIGNS LIST

1 buffer solution
2 ionic buffer solution
3 liquid feeding pump
4 autosampler
5 fractionating column
6 PDA detector
7 fraction collector
8 analysis column
T1 flow-through portion
T2 elution portion using a mixed solution buffer solutions
T3 elution portion using an ionic buffer solution
D drain

1. A method of removing a protein aggregate, comprising:
a step of making a monomer of a protein and an aggregate of proteins adsorb to a column containing a hard porous polymer self-supporting structure to which a strong cation exchange group is fixed by making a solution containing the monomer of the protein and the aggregate of the proteins pass through the column; and
a step of making a mobile phase consisting of a mixed solution of a buffer solution and an ionic buffer solution pass through the column to which the monomer of a protein and the aggregate of proteins are adsorbed, to selectively elute the monomer of a protein.

2. The method of removing a protein aggregate according to claim 1,
wherein the concentration of the buffer solution is 1 mM to 100 mM.

3. The method of removing a protein aggregate according to claim 1,
wherein the pH of the buffer solution is 2 to 9.

4. The method of removing a protein aggregate according to claim 1,
wherein the concentration of inorganic salts in the mixed solution of the buffer solution and the ionic buffer solution is 50 mM to 250 mM.

5. The method of removing a protein aggregate according to claim 1,
wherein monomers constituting the hard porous polymer self-supporting structure are methacrylates or acrylates.

6. The method of removing a protein aggregate according to claim 1,
wherein the thickness of the hard porous polymer self-supporting structure in a direction in which a solution passes through is 1 mm to 100 mm.

7. The method of removing a protein aggregate according to claim 1,
wherein the flow rate of the mobile phase is greater than or equal to 2 CV/minute.
